(19) United States
(12) Patent Application Publication
(10) Pub. No.: US 2017/0347701 A1
(43) Pub. Date: Dec. 7, 2017

(54) METHOD FOR ADMINISTERING TETRA-HYDROCANNABINOL IN TOBACCO

(71) Applicant: Christopher Pitta, Los Angeles, CA (US)

(72) Inventor: Christopher Pitta, Los Angeles, CA (US)

(21) Appl. No.: 15/170,743

(22) Filed: Jun. 1, 2016

(51) Int. Cl. A24D 3/06; A61K 9/00; A24D 3/04; A61K 31/352; A24B 15/16

(52) U.S. Cl. CPC A24D 3/061; A61K 31/352; A24B 15/16; A24D 3/048; A61K 9/007

(57) ABSTRACT

A method for administering tetra-hydrocannabinol in a smokable tobacco product, the method includes providing the smokable tobacco product. The smokable tobacco product is impregnated with an extract of tetra-hydrocannabinol for administering the tetra-hydrocannabinol extract along with fumes from burning tobacco in the smokable tobacco product to a user.

### FIELD OF THE INVENTION

The present invention disclosed herein generally relates to smokable products, such as cigarettes. More particularly, the method disclosed herein relates to a method for administering tetra-hydrocannabinol in tobacco filled in a cigarette.

### BACKGROUND

Traditionally, tetra-hydrocannabinol (THC) has been used as a stimulant and for medical uses due to its pain killing properties. Additionally, THC has been used to reduce nausea and vomiting during chemotherapy, to improve appetite in people with HIV/AIDS, and to treat chronic pain and muscle spasms. Furthermore, THC has been used as a recreational drug due to its ability to induce relaxation and euphoria. Conventionally, THC users use rolling paper to fill THC and prepare a smokable joint similar to a cigarette without a filter member. This inconveniences a user who smokes THC. A THC product similar to the commercially available cigarette, which is readily usable and requires no preparation, is required. Moreover, currently cigarette smokers have the option different kinds of flavors, for example, mint, orange, etc. However, they do not have the option to use double stimulants, for example, tobacco in combination with THC, etc. A product, which contains a double stimulant for enhanced recreational use, is required.

Hence, there is a long felt but unresolved need for a THC product similar to the commercially available cigarette, which is readily usable and requires no preparation. Furthermore, there is a need for a THC product, which contains a double stimulant for enhanced recreational use.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The method disclosed herein addresses the above-mentioned need for a THC product similar to the commercially available cigarette, which is readily usable and requires no preparation. Furthermore, the method disclosed herein addresses the need for a THC product, which contains a double stimulant for enhanced recreational use. The method for administering tetra-hydrocannabinol in a smokable tobacco product, the method includes providing the smokable tobacco product. The smokable tobacco product is impregnated with an extract of tetra-hydrocannabinol for administering the tetra-hydrocannabinol extract along with fumes from burning tobacco in the smokable tobacco product to a user.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates a method for administering tetra-hydrocannabinol in tobacco.

FIG. 2A exemplarily illustrates a perspective view of a cigarette showing a frangible container seated in a filter member of the cigarette.

FIG. 2B exemplarily illustrates a perspective view of a cigarette showing multiple frangible containers positioned on an elongate member.

### DETAILED DESCRIPTION OF THE INVENTION

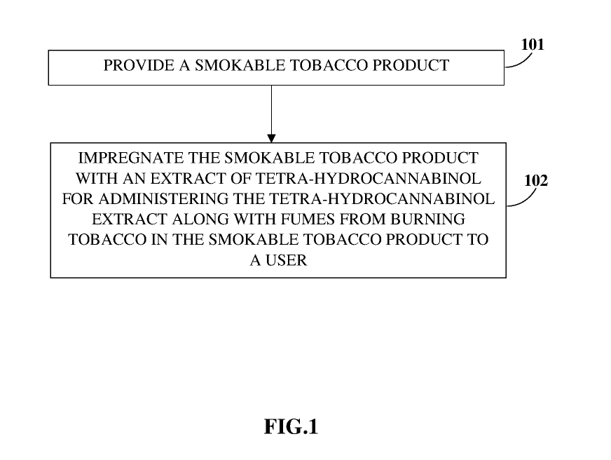

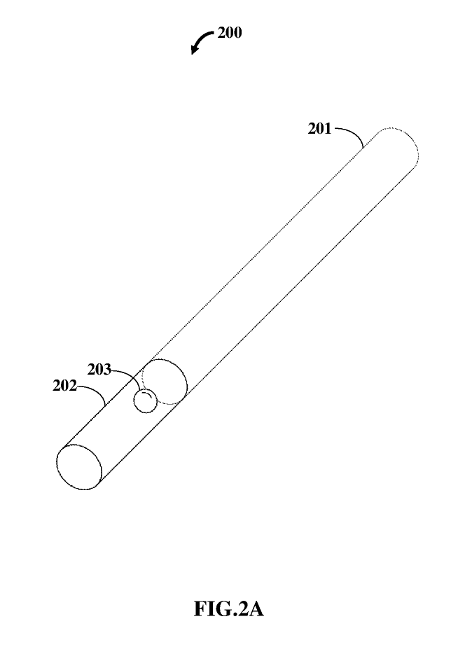

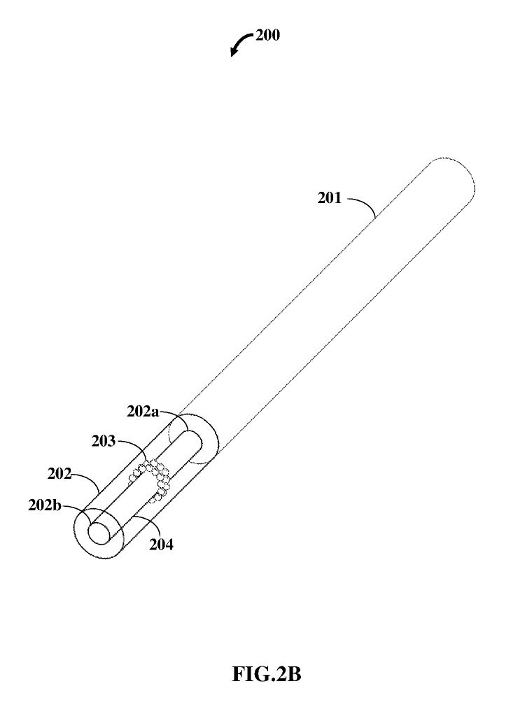

FIG. 1 exemplarily illustrates a method for administering tetra-hydrocannabinol (THC) in tobacco. In the method for administering tetra-hydrocannabinol in a smokable tobacco product disclosed herein, the smokable tobacco product is provided 101. The smokable tobacco product is impregnated 102 with an extract of tetra-hydrocannabinol for administering the tetra-hydrocannabinol extract along with fumes from burning tobacco in the smokable tobacco product to a user. In an embodiment, the smokable tobacco product is, for example, a tobacco pipe. In another embodiment, the smokable tobacco product is a cigarette 200 comprising a tobacco rod 201 and a filter member 202 as exemplarily illustrated in FIGS. 2A-2B. In another embodiment, the smokable tobacco product is impregnated by adding the extract of tetra-hydrocannabinol to the filter member 202 of the smokable tobacco product 200. In another embodiment, the tetra-hydrocannabinol is mixed with the tobacco and inserted into the tobacco rod 201 of the cigarette 200. The impregnated tetra-hydrocannabinol provides additional stimulation to a smoker. The tetra-hydrocannabinol also acts as a relaxant and an analgesic. Furthermore, the tetra-hydrocannabinol reduces pain and prevents inflammatory tendencies.

FIG. 2A exemplarily illustrates a perspective view of a cigarette 200 showing a frangible container 203 seated in a filter member 202 of the cigarette 200. The filter member 202 is typically made of materials, for example, cellulose acetate fiber, paper, activated charcoal, etc. The filter member 202 modifies the particulate smoke phase by particle retention and reduces tar and nicotine smoke yields up to 50 percent. In an embodiment, a frangible container 202 containing an extract of tetra-hydrocannabinol is positioned in the filter member 202 as exemplarily illustrated in FIG. 2A. A user exerting a force on the filter member 202 breaks the fragile frangible container 203 to release the tetra-hydrocannabinol into the filter member 202. The user thus inhales a mixture of tobacco and tetra-hydrocannabinol once he/she lights and smokes the cigarette. The frangible container 203 can be broken at any time based on the preference of the user. For example, the frangible container 203 is broken before lighting for a smoother effect. Additionally, for a stronger dose, the frangible container 203 is broken few moments before the cigarette 200 is extinguished. The user decides when to break the frangible container 203 based on the user's need for more or less of the stimulant's effects. FIG. 2B exemplarily illustrates a perspective view of a cigarette 200 showing multiple frangible containers 203 positioned on an elongate member 204. The elongate member 202 is made of an absorbent material and is attached to a first end 202a and a second end 202b of the filter member 202. In the embodiment illustrated in FIG. 2B, the elongate member 204 absorbs the tetra-hydrocannabinol extract released by the multiple frangible containers 203. The elongate member 204 provides a larger surface contact area for the nicotine smoke from the tobacco rod 201 to contact and absorb the tetra-hydrocannabinol absorbed by the elongate member 204.

What is claimed is:

1. A method for administering tetra-hydrocannabinol in a smokable tobacco product, the method comprising:
providing the smokable tobacco product; and
impregnating the smokable tobacco product with an extract of tetra-hydrocannabinol for administering the tetra-hydrocannabinol extract along with fumes from burning tobacco in the smokable tobacco product to a user.

2. The method of claim 1, wherein the smokable tobacco product is one of a tobacco pipe and an electronic cigarette.

3. The method of claim 1, wherein the smokable tobacco product is a cigarette comprising a tobacco rod and a filter member.

4. The method of claim 3, wherein the filter member comprises at least one frangible container.

5. The method of claim 3, wherein impregnating the smokable tobacco product comprises releasing an extract of tetra-hydrocannabinol from at least one frangible container seated in the filter member for administering the tetra-hydrocannabinol extract along with fumes from burning tobacco in the tobacco rod to a user.

6. The method of claim 4, wherein the at least one frangible container is positioned on an elongate member positioned in the filter member, wherein the elongate member is attached to a first end and a second end of the filter member.

7. The method of claim 6, wherein the elongate member is of an absorbent material and is configured to absorb the tetra-hydrocannabinol extract released from the at least one frangible container.

8. The method of claim 3, wherein impregnating the smokable tobacco product comprises adding an extract of tetra-hydrocannabinol to the filter member of the smokable tobacco product.
